(19) United States
(12) Patent Application Publication
(10) Pub. No.: US 2007/0163602 A1
(43) Pub. Date: Jul. 19, 2007

(54) Color coded restraints

(76) Inventor: Anthony Imperato, Brooklyn, NY (US)

Correspondence Address: SHOEMAKER AND MATTARE, LTD, 10 POST OFFICE ROAD - SUITE 110, SILVER SPRING, MD, 20910, US

(21) Appl. No.: 11/321,083

(22) Filed: Dec. 30, 2005

(51) Int. Cl. A61F 13/00; A61F 5/37

(52) U.S. Cl. 128/846

(57) ABSTRACT

Restraints such as handcuffs of different colors are used to categorize people in custody according to a trait or rating such as propensity to violence. By following a known color coding scheme, custodians can more readily distinguish among the people in custody.

### BACKGROUND OF THE INVENTION

This invention relates to a color coded restraints such as handcuffs.

Groups of prisoners are transferred between facilities in the custody of deputies or other custodians who may have never seen the prisoners before, and cannot evaluate their danger on the basis of appearance. Occasionally, a known violent prisoner may surprise and overpower his custodian, with potentially fatal consequences to the custodian, people nearby, and the prisoner himself. Surprise is more possible if the custodian does not know whom to be particularly careful of.

### SUMMARY OF THE INVENTION

An object of the invention is to enable custodians to distinguish visually different categories of people in their custody.

These and other objects are attained by a color coded restraints as described below.

### BRIEF DESCRIPTION OF THE DRAWINGS

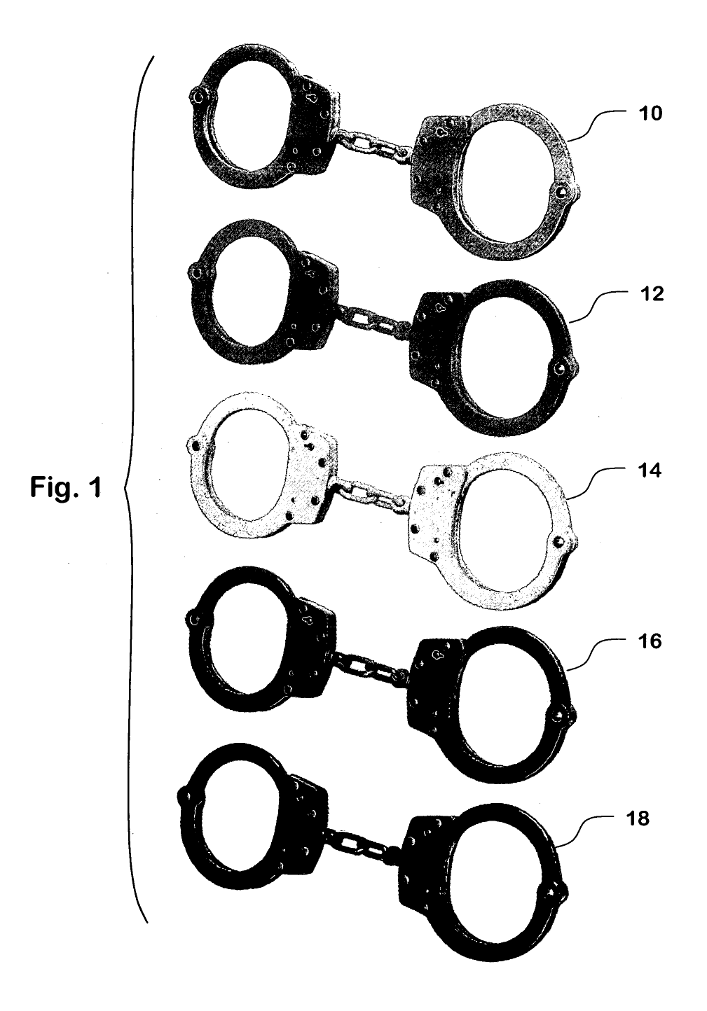

In the accompanying drawings, FIG. 1 is a perspective view of a set of color coded restraints embodying the invention, the various colors being indicated by shading.

### DESCRIPTION OF THE PREFERRED EMBODIMENT

Color coded restraints embodying the invention are exemplified by the handcuffs shown in FIG. 1. Five pairs of handcuffs 10, 12, 14, 16, 18, respectively, are shown. They are of conventional design, made preferably of stainless steel, and are identical except for their color. Each of the handcuffs in the set has a unique color, preferably a bright and visually distinctive color like red, orange, yellow, green or silver (uncolored). Those that are colored have the major surfaces of both cuffs and the connecting chain entirely coated so as to maximize their visual impact and make it impossible for a person to conceal the color by turning the cuffs one way or the other.

The restraints are coated by any known metal coloring means, preferably by powder coating, which produces a durable and stable surface. Colored powders and the processes by which they are applied, are well known and widely available, and therefore are not discussed in detail here. Other coating methods and materials could be used, as long as the material were sufficiently durable, color-fast and harmless to the wearer.

The visually distinct restraints can be used to categorize prisoners according to the nature of their crimes, or perhaps in some other way, such as according to their destination. As an example, red cuffs could be used for violent criminals, orange for a non-violent type offender, and so on. It is anticipated that a country- or world-wide color coding system may arise, and that the system would become universally understood, in much the same way as are traffic light colors or security alert levels.

While the restraints illustrated are handcuffs, other types of restraints could be colored according to the principles of this invention. The restraints do not necessarily have to be made or stainless steel, or even metal. In the case of plastic restraints, the colorant could be mixed into the plastic, rather than being a surface coating.

Since the invention is subject to modifications and variations, it is intended that the foregoing description and the accompanying drawings shall be interpreted as only illustrative of the invention defined by the following claims.

I claim:

1. A set of color coded restraints such as handcuffs in which each restraint within the set is of a different color so that the restraints can be used to categorize people in custody by following a color coding scheme.

2. A set of color coded restraints according to claim 1, wherein substantially the entire outer surface of each restraint is completely and uniformly colored.

3. A set of color coded restraints according to claim 1, wherein each of the restraints within the set is a pair of handcuffs.

4. A method of categorizing people in custody by placing on them restraints of different colors, each color being chosen to correspond to a predetermined category.

5. The method of claim 4, wherein each said category is a category of crime.

6. The method of claim 4, wherein said categories are different levels of propensity to violence.

7. The method of claim 6, wherein the color red is used to denote the highest level of propensity to violence.

8. The method of claim 4, wherein said restraints are handcuffs.
